(19) United States
(12) Patent Application Publication
(10) Pub. No.: US 2021/0030281 A1
(43) Pub. Date: Feb. 4, 2021

(54) Maximum Heart Rate Estimation Method and Device

(71) Applicant: Nippon Telegraph and Telephone Corporation, Tokyo (JP)

(72) Inventors: Yuichi Higuchi, Tokyo (JP); Hiroyoshi Togo, Tokyo (JP); Nobuaki Matsuura, Tokyo (JP)

(21) Appl. No.: 17/046,070

(22) PCT Filed: Apr. 10, 2019
(86) PCT No.: PCT/JP2019/015579

§ 371 Date: Oct. 8, 2020

(30) Foreign Application Priority Data
Apr. 12, 2018 (JP) 2018-076550

(51) Int. Cl. A61B 5/0245; A61B 5/00; A61B 5/11; A61B 5/0472

(52) U.S. Cl. CPC A61B 5/0245; A61B 5/0472; A61B 5/1118; A61B 5/4035

(57) ABSTRACT

A method includes a first acquisition step of acquiring a heart rate of a target person who does exercise, a second acquisition step of acquiring an electrocardiographic waveform of the target person who does the exercise, a third acquisition step of acquiring a predetermined feature amount from the acquired electrocardiographic waveform, and an estimation step of estimating a maximum heart rate of the target person based on a relationship between the predetermined feature amount and the acquired heart rate. In the estimation step, the maximum heart rate of the target person is estimated based on a heart rate corresponding to an inflection point in a change of the predetermined feature amount with respect to the acquired heart rate.

### CROSS-REFERENCE TO RELATED APPLICATIONS

This application is a national phase entry of PCT Application No. PCT/JP2019/015579, filed on Apr. 10, 2019, which claims priority to Japanese Application No. 2018-076550, filed on Apr. 12, 2018, which applications are hereby incorporated herein by reference.

### TECHNICAL FIELD

The present invention relates to a maximum heart rate estimation method and device and, more particularly, to a technique of estimating a maximum heart rate using an electrocardiographic waveform.

### BACKGROUND

In sports and daily life, it is possible to estimate exercise intensity and calories by managing a heart rate. Exercise intensity using a heart rate can generally be calculated by the Karvonen method (see, for example, non-patent literature 1). Therefore, to calculate exercise intensity, it is necessary to measure or estimate the maximum heart rate. Furthermore, the value of exercise intensity is necessary to calculate calories, and it is thus important to measure or estimate the maximum heart rate.

Conventionally, when measuring the maximum heart rate, measurement is performed by increasing momentum until the heart rate of a target person reaches the maximum heart rate by an incremental load test or the like.

### RELATED ART LITERATURE

### Non-Patent Literature

Non-Patent Literature 1: Jinhua She, Hitoshi Nakamura, Koji Makino, Hiroshi Hashimoto, “Selection of Suitable Maximum-heart-rate Formulas for Use with Karvonen Formula to Calculate Exercise Intensity”, International Journal of Automation and Computing, 12(1), February 2015, pp. 62-69.
Non-Patent Literature 2: https://ja.wikipedia.org/wiki/ [symbol] (searched on Mar. 20, 2018)
Non-Patent Literature 3: Shaffer, Fred, and J. P. Ginsberg, “An overview of heart rate variability metrics and norms.” Frontiers in public health 5 (2017): 258
Non-Patent Literature 4: Hideaki Senju, “Chapter 3 Exercise and Change in Cardiopulmonary Function (Part I Regional Wellness)” Health Promotion from Life/Region, Nagasaki University Extension Course Series 7 (published on Mar. 5, 1995): pp. 31-39, http://hdl.handle.net/10069/6315.

### SUMMARY

### Problem to be Solved by Embodiments of the Invention

However, in the conventional method of measuring the maximum heart rate by an incremental load test or the like, the load of a target person is heavy.

Embodiments of the present invention have been made in consideration of the above problem, and has as its object to provide a maximum heart rate estimation method and device capable of estimating the maximum heart rate without requiring exercise to be done until the heart rate of a target person reaches the maximum heart rate.

### Means of Solution to the Problem

In order to solve the above-described problem, a maximum heart rate estimation method according to embodiments of the present invention is a maximum heart rate estimation method comprising a first acquisition step of acquiring a heart rate of a target person who does exercise, a second acquisition step of acquiring an electrocardiographic waveform of the target person who does the exercise, a third acquisition step of acquiring a predetermined feature amount from the acquired electrocardiographic waveform, and an estimation step of estimating a maximum heart rate of the target person based on a relationship between the predetermined feature amount and the acquired heart rate, wherein in the estimation step, the maximum heart rate of the target person is estimated based on a heart rate corresponding to an inflection point in a change of the predetermined feature amount with respect to the acquired heart rate.

According to embodiments of the present invention, there is also provided a maximum heart rate estimation device comprising a heart rate acquisition unit configured to acquire a heart rate of a target person who does exercise, a feature amount acquisition unit configured to acquire an electrocardiographic waveform of the target person who does the exercise, and acquire a predetermined feature amount from the electrocardiographic waveform, and an estimation unit configured to estimate a maximum heart rate of the target person based on a relationship between the acquired heart rate and the predetermined feature amount, wherein the estimation unit estimates the maximum heart rate of the target person based on a heart rate corresponding to an inflection point in a change of the predetermined feature amount with respect to the acquired heart rate.

### Effect of Embodiments of the Invention

According to embodiments of the present invention, since the maximum heart rate is calculated from the relationship between the heart rate of a target person and a predetermined feature amount included in the electrocardiographic waveform of the target person based on an inflection point in a change of the feature amount with respect to the heart rate, it is possible to estimate the maximum heart rate without requiring exercise to be done until the heart rate of the target person reaches the maximum heart rate. Therefore, it is possible to reduce the load of the target person when obtaining the maximum heart rate.

### BRIEF DESCRIPTION OF THE DRAWINGS

FIG. 1 is a view for explaining the principles of embodiments of the present invention;

FIG. 2 is a graph for explaining the principles of a maximum heart rate estimation device according to the first embodiment of the present invention;

FIG. 3 is a graph for explaining the principles of the maximum heart rate estimation device according to the first embodiment of the present invention;

FIG. 4 is a block diagram showing the functional arrangement of the maximum heart rate estimation device according to the first embodiment of the present invention;

FIG. 5 is a block diagram showing the hardware arrangement of the maximum heart rate estimation device according to the first embodiment of the present invention;

FIG. 6 is a flowchart for explaining a maximum heart rate estimation method according to the first embodiment of the present invention;

FIG. 7 is a graph for explaining the principles of a maximum heart rate estimation device according to the second embodiment of the present invention;

FIG. 8 is a block diagram showing the functional arrangement of the maximum heart rate estimation device according to the second embodiment of the present invention;

FIG. 9 is a flowchart for explaining a maximum heart rate estimation method according to the second embodiment of the present invention;

FIG. 10 is a graph for explaining the principles of a maximum heart rate estimation device according to the third embodiment of the present invention;

FIG. 11 is a block diagram showing the functional arrangement of the maximum heart rate estimation device according to the third embodiment of the present invention; and

FIG. 12 is a flowchart for explaining a maximum heart rate estimation method according to the third embodiment of the present invention.

### DETAILED DESCRIPTION OF ILLUSTRATIVE EMBODIMENTS

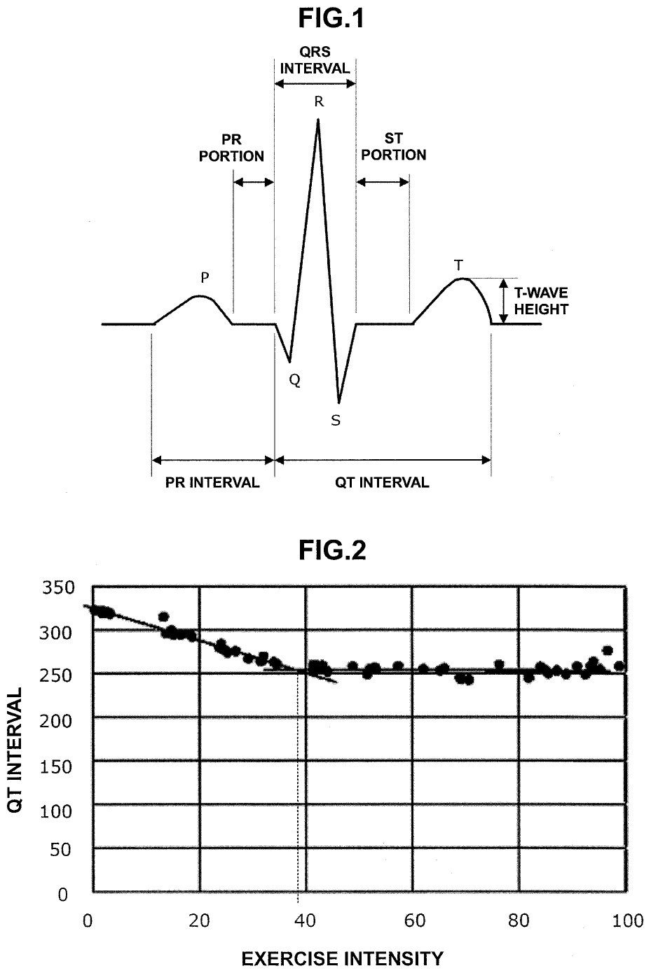

Preferred embodiments of the present invention will be described in detail below with reference to FIGS. 1 to 12.

### Principles of Embodiments of Invention

FIG. 1 is a view showing an electrocardiographic waveform. A maximum heart rate estimation method according to an embodiment of the present invention extracts a predetermined feature amount serving as an index of the maximum heart rate from the electrocardiographic waveform of a target person, and estimates, for example, the maximum heart rate for obtaining exercise intensity. The exercise intensity is a scale representing the vigorousness of exercise with reference to the physical ability of the target person who does the exercise.

In the maximum heart rate estimation method according to this embodiment, among characteristic waveforms and waveform intervals included in the electrocardiographic waveform shown in FIG. 1, a QT interval representing the time from the start of a Q wave to the end of a T wave is used as a feature amount of the electrocardiographic waveform (electrocardiogram).

FIG. 2 is a graph showing the relationship between a QT interval and exercise intensity acquired by an incremental load test. In this embodiment, as the exercise intensity, a value calculated from a heart rate (HR) is used. As shown in FIG. 2, it is apparent that there is an inflection point in the graph around a portion where the exercise intensity is 40%. This experiment indicates that a heart rate at the inflection point corresponds to exercise intensity of about 40%.

This experiment result is considered to coincide with the relationship between a change in cardiac output and a stroke volume by exercise described in non-patent literature 4. In general, the oxygen demand of a peripheral tissue such as a muscle is increased by exercise. As described in non-patent literature 4, it is known that the stroke volume (SV) as a blood volume ejected by one contraction of the heart increases at a predetermined rate along with an increase in exercise intensity but reaches a plateau without increasing after the exercise intensity increases to about 40% of the maximum exercise intensity. Therefore, in exercise in which the exercise intensity exceeds about 40%, the cardiac output (CO) as a blood volume ejected from the heart per minute is supplemented by increasing the heart rate.

From this viewpoint, it is considered that it is possible to estimate the maximum heart rate using the above-described experiment result by setting the heart rate at the inflection point of the QT interval to correspond to exercise intensity of 40%.

At this time, the maximum heart rate is calculated (see, for example, non-patent literature 2) by:

maximum heart rate=resting heart rate+100×(heart rate at inflection point−resting heart rate)/exercise intensity at inflection point (1)

In equation (1) above, “resting heart rate” is a value measured in advance at the time of rest by a heart rate meter or the like. In general, 60 bpm is used as the resting heart rate. “Heart rate at inflection point” is the value of a measured heart rate corresponding to the inflection point of the QT interval. In addition, “exercise intensity at inflection point” is a preset value, and a value falling within the range of 35% to 45%, for example, 40% is used based on the above-described experiment result.

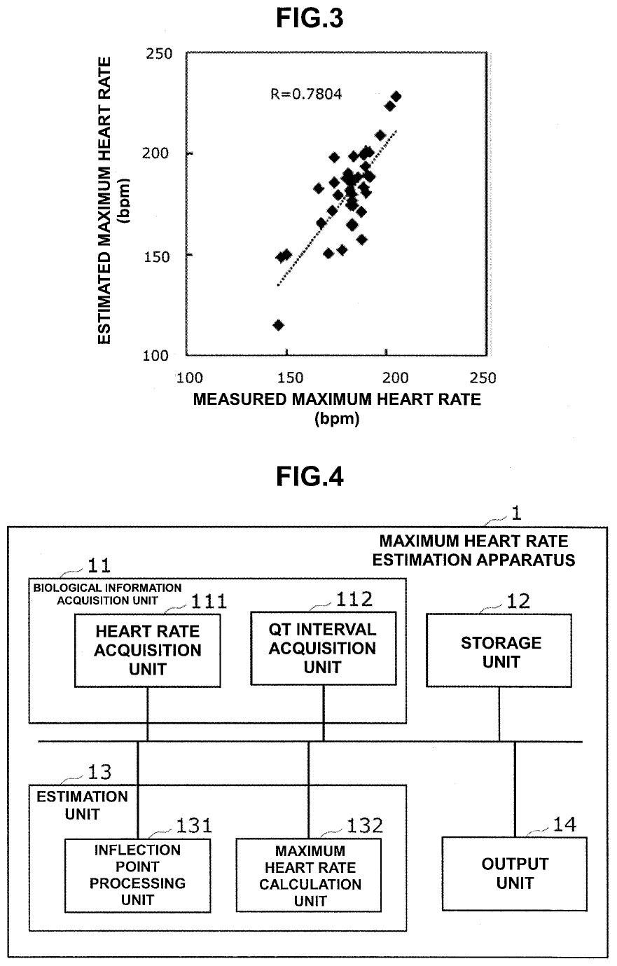

FIG. 3 is a graph showing a result when estimating the maximum heart rate using equation (1) above. In FIG. 3, the ordinate represents the maximum heart rate estimated by the maximum heart rate estimation method according to this embodiment and the abscissa represents the maximum heart rate measured by doing exercise until the heart rate of the target person reaches the maximum heart rate.

As shown in FIG. 3, since a correlation coefficient (Pearson correlation function) is 0.78 and p value <0.05 is satisfied, it is found that there is a significant positive correlation. Therefore, the maximum heart rate estimation method according to this embodiment finds the inflection point of the QT interval in the relationship between the QT interval and the heart rate, and calculates the maximum heart rate using equation (1) above.

### First Embodiment

A maximum heart rate estimation device 1 for executing a maximum heart rate estimation method according embodiments of to the present invention will be described in detail below.

FIG. 4 is a block diagram showing the functional arrangement of the maximum heart rate estimation device 1 according to the first embodiment. The maximum heart rate estimation device 1 includes a biological information acquisition unit 11, a storage unit 12, an estimation unit 13, and an output unit 14.

The maximum heart rate estimation device 1 acquires the relationship between a QT interval and a heart rate when a target person does exercise like an incremental load test that gradually increases exercise intensity, extracts the inflection point of the value of QT interval, and calculates the maximum heart rate of the target person from the inflection point.

The biological information acquisition unit 11 includes a heart rate acquisition unit 111 and a QT interval acquisition unit 112.

The biological information acquisition unit 11 acquires information concerning the heart beats and electrocardiogram of the target person from an external biological sensor (not shown) having the functions of the heart rate meter and electrocardiograph and attached to the target person. At this time, the biological information acquisition unit 11 acquires, from the above-described experiment result, information concerning the heartbeats and electrocardiogram for a period from when the target person starts exercise that gradually increases the exercise intensity until the exercise intensity exceeds 40%.

The heart rate acquisition unit 111 acquires, from the biological sensor attached to the target person, a heart rate for a period during which the target person does exercise. Data of the acquired heart rate is stored in the storage unit 12.

The QT interval acquisition unit 112 acquires a QT interval included in the electrocardiographic waveform from the electrocardiographic waveform measured by the biological sensor attached to the target person. The QT interval acquired by the QT interval acquisition unit 112 is stored in the storage unit 12.

The storage unit 12 stores data of the heart rate and the QT interval of the target person acquired by the biological information acquisition unit 11. Furthermore, the storage unit 12 stores setting values of “resting heart rate” and “exercise intensity at inflection point” in equation (1) above.

The estimation unit 13 includes an inflection point processing unit 131 and a maximum heart rate calculation unit 132.

The estimation unit 13 estimates the maximum heart rate based on the data of the heart rate and the QT interval of the target person acquired by the biological information acquisition unit 11.

The inflection point processing unit 131 reads out, from the storage unit 12, the data of the heart rate of the target person acquired by the heart rate acquisition unit 111 and the data of and the QT interval of the target person acquired by the QT interval acquisition unit 112, and obtains the relationship between the QT interval and the heart rate. At this time, the graph including the inflection point shown in FIG. 2 is obtained.

Furthermore, the inflection point processing unit 131 extracts, from the relationship between the QT interval and the heart rate of the target person, an inflection point in a change of the QT interval with respect to the heart rate acquired by the heart rate acquisition unit 111. The inflection point processing unit 131 obtains the heart rate of the target person corresponding to the inflection point of the value of QT interval, and stores it in the storage unit 12.

The maximum heart rate calculation unit 132 calculates the maximum heart rate of the target person by equation (1) above based on the heart rate of the target person at the inflection point extracted by the inflection point processing unit 131.

More specifically, the maximum heart rate calculation unit 132 substitutes, into equation (1), the value of “heart rate at inflection point” obtained by the inflection point processing unit 131. Note that in equation (1), a preset value, for example, 60 bpm is used as the value of “resting heart rate” and a preset value, for example, 40% is used as the value of “exercise intensity at inflection point”.

The maximum heart rate calculation unit 132 stores the calculated value of the maximum heart rate of the target person in the storage unit 12.

The output unit 14 outputs information such as the maximum heart rate of the target person estimated by the estimation unit 13. More specifically, the output unit 14 displays the value of the maximum heart rate calculated by the maximum heart rate calculation unit 132 on a display screen or the like.

### Hardware Arrangement of Maximum Heart Rate Estimation Device

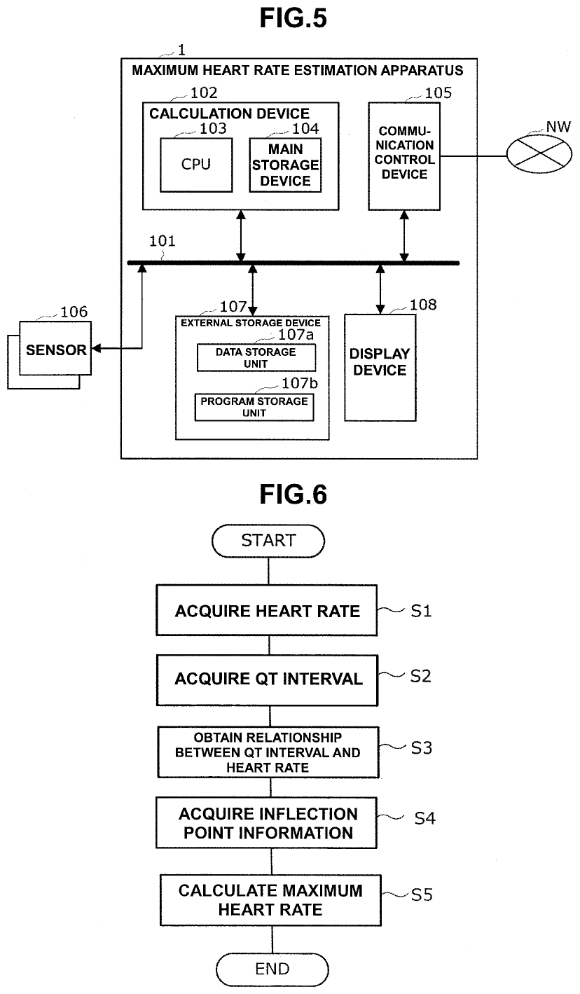

The hardware arrangement of the maximum heart rate estimation device 1 having the above-described functional arrangement will be described next with reference to a block diagram shown in FIG. 5.

As shown in FIG. 5, the maximum heart rate estimation device 1 can be implemented by a computer including a calculation device 102 with a CPU 103 and a main storage device 104, a communication control device 105, a sensor 106, an external storage device 107, and a display device 108, all of which are connected via a bus 101, and a program for controlling these hardware resources.

The CPU 103 and the main storage device 104 form the calculation device 102. A program used by the CPU 103 to perform various control and calculation operations is stored in advance in the main storage device 104. The calculation device 102 implements the functions of the maximum heart rate estimation device 1 including the estimation unit 13 shown in FIG. 4.

The communication control device 105 is a control device for connecting the maximum heart rate estimation device 1 and various external electronic devices by a communication network NW. The communication control device 105 may receive, via the communication network NW, the data of the heart rate and electrocardiograph waveform from the sensor 106 (to be described later) attached to the target person.

The sensor 106 is implemented by, for example, a biological sensor such as a heart rate meter and an electrocardiograph. The sensor 106 is attached to, for example, the chest or wrist of the target person for a period during which the target person does exercise, and measures the heart rate and the electrocardiographic waveform of the target person. For example, the sensor 106 attached to the chest measures the electrocardiographic waveform by an electrode (not shown), and detects heartbeats from a change of the electrocardiographic waveform, thereby measuring, as a heart rate, a heartbeat count per minute from an interval between the heartbeats.

The external storage device 107 is formed by a readable/writable storage medium and a driving device for reading/writing various kinds of information such as programs and data from/in the storage medium. For the external storage device 107, a hard disk or a semiconductor memory such as a flash memory can be used as a storage medium. The external storage device 107 can include a data storage unit 107a, a program storage unit 107b, and another storage device (not shown), for example, a storage device for backing up the programs and data stored in the external storage device 107.

The data storage unit 107a stores information concerning the electrocardiographic waveform and the heart rate of the target person measured by the sensor 106. The data storage unit 107a corresponds to the storage unit 12 shown in FIG. 4.

The program storage unit 107b stores various programs for executing processing necessary to estimate the maximum heart rate, such as processing of acquiring the heart rate and QT interval, inflection point processing, and maximum heart rate calculation processing, according to this embodiment.

The display device 108 forms the display screen of the maximum heart rate estimation device 1, and functions as the output unit 14. The display device 108 is implemented by a liquid crystal display or the like.

### Operation of Maximum Heart Rate Estimation Device

The operation of the maximum heart rate estimation device 1 for executing the above-described maximum heart rate estimation method according to embodiments of the present invention will be described next with reference to a flowchart shown in FIG. 6. First, a biological sensor (not shown) having the functions of the heart rate meter and electrocardiograph is attached to the chest or wrist of a target person, and the target person starts exercise like an incremental load test that gradually increases exercise intensity. The biological sensor measures the heart rate and electrocardiographic waveform of the target person for a period from when the target person starts exercise until the exercise intensity of the target person exceeds 40%.

The heart rate acquisition unit 111 acquires heart rate data while the target person does the exercise (step S1). Next, the QT interval acquisition unit 112 acquires electrocardiographic waveform data while the target person does the exercise, and acquires QT interval data from the electrocardiographic waveform data (step S2). Note that the QT interval acquisition unit 112 may adopt an arrangement of acquiring the value of a QT interval obtained by the external biological sensor.

Next, the inflection point processing unit 131 obtains the relationship between the acquired QT interval and heart rate (step S3). In the relationship between the QT interval and the heart rate of the target person, which has been obtained by the inflection point processing unit 131, the inflection point of the value of the QT interval is extracted, and a heart rate corresponding to the value of the QT interval is obtained (step S4).

Next, the maximum heart rate calculation unit 132 calculates the maximum exercise intensity of the target person using equation (1) above based on the heart rate at the inflection point of the measured value of the QT interval obtained in step S4 (step S5).

More specifically, the maximum heart rate calculation unit 132 uses, in equation (1), a value actually measured as the value of “resting heart rate”, for example, 60 bpm. The maximum heart rate calculation unit 132 substitutes, as the value of “heart rate at inflection point”, the measured value of the heart rate obtained in step S4. Furthermore, a value falling within the predetermined range of 35% to 45%, for example, 40% is used as the value of “exercise intensity at the inflection point” from the above-described experiment result.

Note that the output unit 14 outputs the calculated maximum exercise intensity of the target person.

As described above, according to the first embodiment, the maximum heart rate is estimated based on the heart rate of the target person at the inflection point of the QT interval around a portion where the exercise intensity (heart rate) is 40% using the relationship between the QT interval and the heart rate of the target person. Therefore, if exercise is done until the exercise intensity exceeds 40%, it is possible to estimate the maximum heart rate without requiring exercise to be done until the heart rate of the target person reaches the maximum heart rate. Thus, it is possible to reduce the load of the target person when obtaining the maximum heart rate.

Furthermore, the above-described first embodiment has explained the case in which the QT interval is acquired from the measured electrocardiographic waveform of the target person. However, an RT interval as the time from the start of an R wave to the end of a T wave included in the electrocardiographic waveform shown in FIG. 1 may be used as a feature amount of the electrocardiographic waveform, instead of the QT interval. It is possible to acquire a feature amount corresponding to the QT interval more easily using the RT interval.

### Second Embodiment

The second embodiment of the present invention will be described next. Note that in the following description, the same reference numerals as those in the above-described first embodiment denote similar components and a repetitive description thereof will be omitted.

In the first embodiment, a heart rate corresponding to the inflection point of the value of the QT interval around a portion where the exercise intensity (heart rate) is about 40% is obtained based on the relationship between the QT interval and heart rate measured for the period during which the target person does exercise, thereby calculating the maximum heart rate of the target person. To the contrary, in the second embodiment, the height of a T wave included in the electrocardiographic waveform of a target person is used as a feature amount of the electrocardiographic waveform.

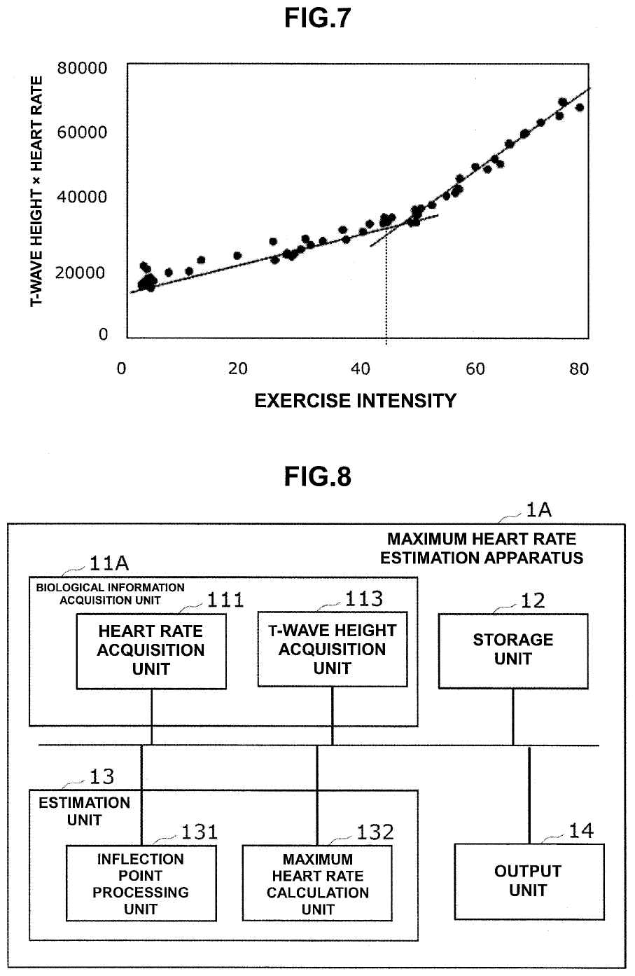

An overview of a maximum heart rate estimation device 1A according to the second embodiment will be described with reference to FIG. 7. In a graph shown in FIG. 7, the abscissa represents exercise intensity obtained by heart rate conversion, and the ordinate represents a value (T-wave height×heart rate) obtained by multiplying the height of a T wave in the electrocardiographic waveform of a target person by a heart rate.

As shown in FIG. 7, in “T-wave height of electrocardiographic waveform×heart rate” as well, there may be an inflection point around a portion where the exercise intensity (heart rate) is 40%. Therefore, it is possible to calculate the maximum heart rate of the target person using equation (1) above, similar to the first embodiment, by obtaining a measured heart rate corresponding to the inflection point.

With respect to the functional arrangement of the maximum heart rate estimation device 1A according to this embodiment, components different from those in the first embodiment will mainly be described next.

A biological information acquisition unit 11A includes a heart rate acquisition unit 111 and a T-wave height acquisition unit 113.

The T-wave height acquisition unit 113 acquires an electrocardiographic waveform from a biological sensor attached to a target person, and acquires data of a T-wave height included in the electrocardiographic waveform. The data of the T-wave height acquired by the T-wave height acquisition unit 113 is stored in a storage unit 12.

An inflection point processing unit 131 calculates “T-wave height×heart rate” by multiplying the value of the T-wave height acquired by the T-wave height acquisition unit 113 of the biological information acquisition unit 11A by the value of the heart rate of the target person acquired by a heart rate acquisition unit 111. Furthermore, the inflection point processing unit 131 obtains the relationship between “T-wave height×heart rate” and the measured value of the heart rate of the target person acquired by the heart rate acquisition unit 111.

The inflection point processing unit 131 finds the inflection point of the value of “T-wave height×heart rate” around a portion where the exercise intensity (heart rate) is 40% in the obtained relationship between the heart rate and “T-wave height×heart rate” of the target person. The inflection point processing unit 131 obtains a heart rate corresponding to the inflection point. The value of the obtained heart rate is stored in the storage unit 12.

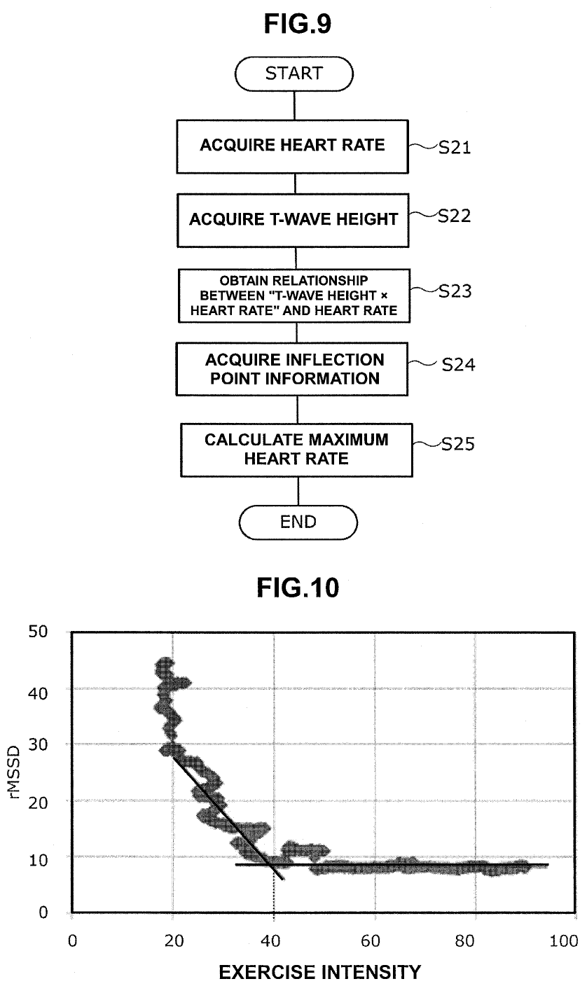

The operation of the maximum heart rate estimation device 1A having the above-described arrangement will be described next with reference to a flowchart shown in FIG. 9.

Similar to the first embodiment, first, a biological sensor (not shown) having the functions of a heart rate meter and electrocardiograph is attached to the chest or wrist of the target person, and the target person starts exercise like an incremental load test that gradually increases exercise intensity. The biological sensor measures the heart rate and electrocardiographic waveform of the target person for a period from when the target person starts the exercise until the exercise intensity of the exercise done by the target person exceeds 40%.

The heart rate acquisition unit 111 acquires heart rate data for a period during which the target person does the exercise (step S21). Next, the T-wave height acquisition unit 113 acquires T-wave height data from electrocardiographic waveform data for the period during which the target person does the exercise (step S22). Note that the T-wave height acquisition unit 113 may adopt an arrangement of acquiring T-wave height data obtained on the biological sensor side.

The inflection point processing unit 131 obtains the relationship between the acquired heart rate and “T-wave height×heart rate” (step S23). After that, the inflection point processing unit 131 extracts the inflection point of the value of “T-wave height×heart rate” in the obtained relationship between the heart rate and “T-wave height×heart rate”, and obtains the value of a heart rate corresponding to the value of “T-wave height×heart rate” (step S24).

Next, a maximum heart rate calculation unit 132 calculates the maximum exercise intensity of the target person using equation (1) above based on the heart rate at the inflection point of the value of “T-wave height×heart rate” obtained in step S24 (step S25).

More specifically, the maximum heart rate calculation unit 132 uses, in equation (1), a value actually measured as the value of “resting heart rate”, for example, 60 bpm. The maximum heart rate calculation unit 132 substitutes, as the value of “heart rate at inflection point”, the heart rate obtained in step S24. Furthermore, a predetermined value, for example, a value such as 40% falling within the range of 35% to 45% is used as the value of “exercise intensity at the inflection point” from the above-described experiment result.

Note that an output unit 14 outputs the calculated maximum exercise intensity of the target person.

As described above, according to the second embodiment, the maximum heart rate is estimated based on the heart rate of the target person at the inflection point of “T-wave height×heart rate” around a portion where the exercise intensity (heart rate) is 40% using the relationship between the heart rate and “T-wave height×heart rate” of the target person. Therefore, even when the T-wave height is used as a feature amount of the electrocardiographic waveform, if exercise is done until the exercise intensity exceeds 40%, it is possible to estimate the maximum heart rate without requiring exercise to be done until the heart rate of the target person reaches the maximum heart rate. Thus, it is possible to reduce the load of the target person when obtaining the maximum heart rate.

### Third Embodiment

The third embodiment of the present invention will be described next. Note that in the following description, the same reference numerals as those in the above-described first and second embodiments denote similar components and a repetitive description thereof will be omitted.

In the first and second embodiments, based on the relationship between the heart rate measured for the period during which the target person does the exercise and a feature amount such as the QT interval or T-wave height observed in the electrocardiographic waveform, the maximum heart rate of the target person is calculated from the heart rate corresponding to the inflection point of the feature amount around a portion where the exercise intensity is about 40%. To the contrary, in the third embodiment, rMSSD (Root Mean Square of Successive Differences) as a heart rate variability parameter known as an autonomic nerve index is used as a feature amount.

First, an overview of a maximum heart rate estimation method according to the third embodiment will be described with reference to FIG. 1.

FIG. 10 is a graph for explaining the relationship between rMSSD and exercise intensity. The ordinate represents rMSSD and the abscissa represents exercise intensity obtained by heart rate conversion.

Note that rMSSD is the root mean square of successive differences between neighboring R-R intervals in an electrocardiographic waveform, and is known as an index concerning autonomic nerves. As is apparent from FIG. 10, in the relationship between rMSSD and the exercise intensity as well, there is the inflection point of the value of rMSSD around a portion where the exercise intensity is 40%.

Therefore, the maximum heart rate of the target person is calculated by equation (1) above using the rMSSD value as the feature amount of the electrocardiographic waveform.

Next, with respect to the functional arrangement of a maximum heart rate estimation device 1B for executing a maximum heart rate estimation method according to this embodiment, components different from those in the first and second embodiments will mainly be described.

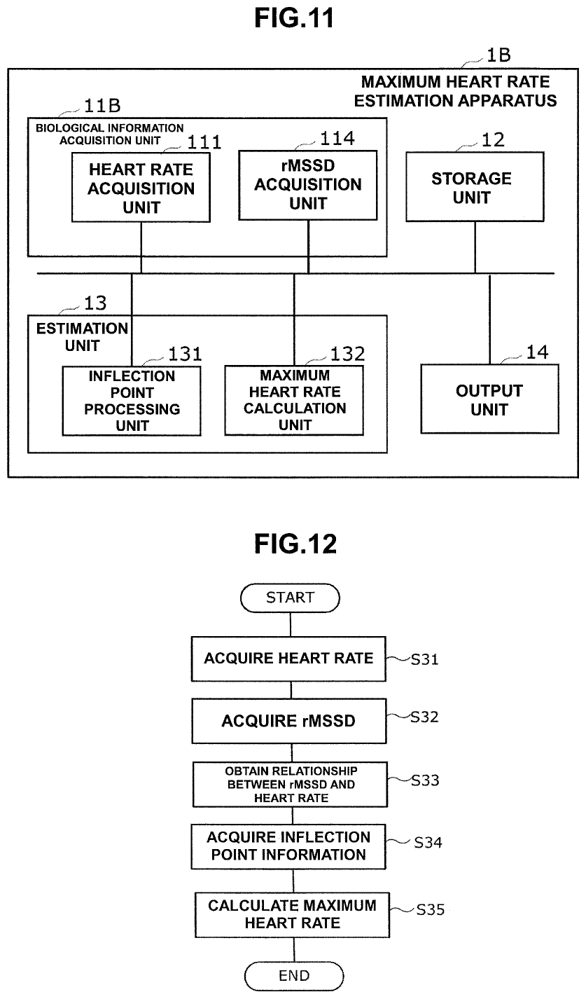

As shown in FIG. 11, a biological information acquisition unit 11B includes a heart rate acquisition unit 111 and an rMSSD acquisition unit 114.

The rMSSD acquisition unit 114 acquires an electrocardiographic waveform from a biological sensor attached to a target person, and acquires data of neighboring R-R intervals included in the electrocardiographic waveform. The rMSSD acquisition unit 114 calculates rMSSD based on the acquired data indicating the R-R intervals. The rMSSD value of the target person calculated by the rMSSD acquisition unit 114 is stored in a storage unit 12.

An inflection point processing unit 131 obtains the relationship between the heart rate of the target person acquired by the heart rate acquisition unit 111 and rMSSD of the target person acquired by the rMSSD acquisition unit 114. The inflection point processing unit 131 extracts the inflection point of the rMSSD value in the obtained relationship between the heart rate and rMSSD of the target person. The inflection point processing unit 131 obtains a heart rate corresponding to the inflection point. The value of the obtained heart rate is stored in the storage unit 12.

The operation of the maximum heart rate estimation device 1B having the above-described arrangement will be described next with reference to a flowchart shown in FIG. 12.

Similar to the first and second embodiments, first, the heart rate acquisition unit 111 acquires the measured value of a heart rate for a period from when the target person does exercise like an incremental load test that gradually increases exercise intensity until the exercise intensity exceeds 40% (step S31).

Next, the rMSSD acquisition unit 114 acquires data indicating the R-R intervals from an electrocardiographic waveform sent from the biological sensor attached to the target person, and calculates rMSSD (step S32). Note that the rMSSD acquisition unit 114 may adopt an arrangement of acquiring an rMSSD value calculated by the external biological sensor.

Next, the inflection point processing unit 131 obtains the relationship between the heart rate of the target person acquired by the heart rate acquisition unit 111 and rMSSD of the target person acquired by the rMSSD acquisition unit 114 (step S33).

After that, the inflection point processing unit 131 finds the inflection point of the rMSSD value in the obtained relationship between the heart rate and rMSSD of the target person, and obtains the value of a heart rate corresponding to the rMSSD value (step S34).

Next, a maximum heart rate calculation unit 132 calculates the maximum exercise intensity of the target person using equation (1) above based on the heart rate at the inflection point of the rMSSD value obtained in step S34 (step S35).

More specifically, the maximum heart rate calculation unit 132 uses, in equation (1), a value actually measured as the value of “resting heart rate”, for example, 60 bpm. The maximum heart rate calculation unit 132 substitutes, as the value of “heart rate at inflection point”, the heart rate obtained in step S34. Furthermore, a preset value, for example, a value such as 40% falling within the range of 35% to 45% is used as the value of “exercise intensity at the inflection point” from the above-described experiment result.

Note that an output unit 14 outputs the calculated maximum exercise intensity of the target person.

As described above, according to the third embodiment, the maximum heart rate is estimated based on the heart rate of the target person at the inflection point of rMSSD around a portion where the exercise intensity is 40% using the relationship between the heart rate and rMSSD of the target person. Therefore, even when rMSSD is used as a feature amount in the electrocardiographic waveform, if exercise is done until the exercise intensity exceeds 40%, it is possible to estimate the maximum heart rate without requiring exercise to be done until the heart rate of the target person reaches the maximum heart rate. Thus, it is possible to reduce the load of the target person when obtaining the maximum heart rate.

This embodiment estimates the maximum heart rate of the target person by paying attention to rMSSD serving as the autonomic nerve index but can use another autonomic nerve index instead of rMSSD. Therefore, it is possible to estimate the maximum heart rate with higher flexibility. Other examples of the autonomic nerve index are an LF value, HF value, and LF/HF ratio in the frequency domain and SDNN (Standard Deviation of the NN intervals), NN50, and pNN50 in the time domain (see, for example, non-patent literature 3).

The embodiments in the maximum heart rate estimation method and the maximum heart rate estimation device according to the present invention have been described above. However, the present invention is not limited to the above-described embodiments, and various modifications conceivable by those skilled in the art can be made within the scope of the invention described in the claims.

Note that each of the above-described embodiments has explained the case in which the target person does exercise like an incremental load test and data concerning a heart rate and an electrocardiographic waveform for an exercise period is acquired. However, the exercise done by the target person need not be managed exercise such as the incremental load test. The maximum heart rate may be estimated by acquiring the relationship between a heart rate and a feature amount such as a QT interval from arbitrary exercise data and obtaining a heart rate at an inflection point.

### EXPLANATION OF THE REFERENCE NUMERALS AND SIGNS

1, 1A, 1B . . . maximum heart rate estimation device, 11, 11A, 11B . . . biological information acquisition unit, 12 . . . storage unit, 13 . . . estimation unit, 14 . . . output unit, 111 . . . heart rate acquisition unit, 112 . . . QT interval acquisition unit, 113 . . . T-wave height acquisition unit, 114 . . . rMSSD acquisition unit, 131 . . . inflection point processing unit, 132 . . . maximum heart rate calculation unit, 101 . . . bus, 102 . . . calculation device, 103 . . . CPU, 104 . . . main storage device, 105 . . . communication control device, 106 . . . sensor, 107 . . . external storage device, 107a . . . data storage unit, 107b . . . program storage unit, 108 . . . display device, NW . . . communication network.

1.-8. (canceled)

9. A maximum heart rate estimation method comprising:
acquiring a first heart rate of a target person who does exercise;
acquiring an electrocardiographic waveform of the target person;
acquiring a predetermined feature amount from the electrocardiographic waveform; and
estimating a maximum heart rate of the target person based on a relationship between the predetermined feature amount and the first heart rate, wherein the maximum heart rate of the target person is estimated based on a second heart rate corresponding to an inflection point in a change of the predetermined feature amount with respect to the first heart rate.

10. The maximum heart rate estimation method according to claim 9, wherein the first heart rate and the electrocardiographic waveform are acquired for a period from when the target person starts the exercise until an exercise intensity exceeds 40%, and wherein the exercise intensity increases from when the target person starts the exercise.

11. The maximum heart rate estimation method according to claim 9, wherein the predetermined feature amount is a QT interval included in the electrocardiographic waveform.

12. The maximum heart rate estimation method according to claim 9, wherein the predetermined feature amount is a height of a T wave included in the electrocardiographic waveform.

13. The maximum heart rate estimation method according to claim 9, wherein the predetermined feature amount is an index concerning autonomic nerves included in the electrocardiographic waveform.

14. The maximum heart rate estimation method according to claim 9, wherein estimating the maximum heart rate comprises estimating the maximum heart rate according to:
the maximum heart rate=a resting heart rate+100×(the second heart rate−the resting heart rate)/an exercise intensity at the inflection point,
wherein the resting heart rate is a heart rate measured in advance at a time of rest, and the exercise intensity at the inflection point is a preset value).

15. A maximum heart rate estimation device comprising:
one or more processors; and
a non-transitory computer-readable storage medium storing a program to be executed by the one or more processors, the program including instructions for:
acquiring a first heart rate of a target person who does exercise;
acquiring an electrocardiographic waveform of the target person;
acquiring a predetermined feature amount from the electrocardiographic waveform; and
estimating a maximum heart rate of the target person based on a relationship between the first heart rate and the predetermined feature amount, wherein the maximum heart rate of the target person is estimated based on a second heart rate corresponding to an inflection point in a change of the predetermined feature amount with respect to the first heart rate.

16. The maximum heart rate estimation device according to claim 15, wherein the first heart rate and the electrocardiographic waveform are acquired for a period from when the target person starts the exercise until an exercise intensity exceeds 40%, and wherein the exercise intensity increases from the target person starts the exercise.

17. The maximum heart rate estimation device according to claim 15, wherein the predetermined feature amount is a QT interval included in the electrocardiographic waveform.

18. The maximum heart rate estimation device according to claim 15, wherein the predetermined feature amount is a height of a T wave included in the electrocardiographic waveform.

19. The maximum heart rate estimation device according to claim 15, wherein the predetermined feature amount is an index concerning autonomic nerves included in the electrocardiographic waveform.

20. The maximum heart rate estimation device according to claim 15, wherein the maximum heart rate is estimated according to:
the maximum heart rate=a resting heart rate+100×(the second heart rate−the resting heart rate)/an exercise intensity at the inflection point,
wherein the resting heart rate is a heart rate measured in advance at a time of rest, and wherein the exercise intensity at the inflection point is a preset value.
